(19) United States
(12) Patent Application Publication
(10) Pub. No.: US 2016/0024462 A1
(43) Pub. Date: Jan. 28, 2016

(54) METHOD AND DEVICE FOR PRODUCING A CELL CULTURE OF HUMAN OR ANIMAL CELLS

(71) Applicant: XELLUTEC GMBH, Neuried (DE)

(72) Inventor: Günter Bertholdt, Germering (DE)

(73) Assignee: XELLUTEC GMBH, Neuried (DE)

(21) Appl. No.: 14/778,664

(22) PCT Filed: Mar. 25, 2014
(86) PCT No.: PCT/EP2014/055940

§ 371 Date: Sep. 21, 2015

(30) Foreign Application Priority Data
Mar. 25, 2013 (DE) 102013005198.3

(51) Int. Cl. C12N 5/00; C12M 1/12; C12M 1/32

(52) U.S. Cl. CPC C12N 5/0068; C12M 23/12; C12M 37/04; C12N 2533/78

(57) ABSTRACT

The invention relates to a method for producing a cell culture (9) of human or animal cells, in particular mammalian cells, for the purpose of carrying out medical or pharmacological assays. According to the invention, the method comprises at least the following steps: inserting a substrate (3) of microbially produced cellulose into a vessel (1); introducing a nutrient solution (2) into the vessel (1); applying human or animal cells (4) to the substrate (3); conditioning the vessel contents to approximately body temperature; and waiting until the substrate (3) is completely covered by a cell lawn (5), i.e. confluence has been reached. Medical or pharmacological assays can then be carried out on the cell culture (9) thus produced.

### PRIOR ART

The invention relates to a method for producing a cell culture of human or animal cells according to the preamble of claim 1, a microwell plate having several wells which are filled with a cell culture according to the preamble of claim 12, and a use of a cell culture according to the preamble of claim 14.

Medical or pharmacological assays in which the effect of certain active substances is investigated are, as a rule, carried out on cell cultures of human or animal mammalian cells. The cell culture is therein exposed to the substances to be tested and the reaction of the cells is investigated.

For medical and pharmacological assays, today, as a rule, cell cultures are used in which the cells adhere to a surface, such as, for example, to the base of a cell culture vessel. For this, a suspension of human or animal cells is pipetted into a vessel in which a nutrient solution is contained. The cells then sediment in the nutrient solution and drop to the base of the vessel. They attach there and divide at a division rate which is substantially dependent on the growth factors contained in the nutrient solution. After a few days, a so-called cell lawn forms on the base of the vessel which completely covers the base. This cell lawn is, however, only stable for a limited time. If the cells remain in the vessel for too long—as a rule after a few days—the cells lift from the base of the vessel and die. It is therefore necessary to carry out a so-called passage at regular intervals in which the cells are detached from the vessel base by means of an enzyme, washed, separated and then pipetted into another vessel for new cell division. This process is called passaging and is usually carried out by hand. It is relatively time-consuming and expensive. Additionally, the cells change after each passage—their phenotype as well as their specific properties. In other words, they dedifferentiate and develop from a differentiated state with very specific properties, such as, for example, skin cells or liver cells, into unspecific cells which are ultimately unsuitable for investigations. In principle it applies that cells of a younger passage (daughter generation) are usually less differentiated than cells of an older passage (parent generation). After several passages, the cells contained in the suspension eventually have completely different properties from the original initial cells.

Consequently, the same medical assay which is carried out once on a cell culture of an older and once on a cell culture of a younger generation can give different results. Therefore, assays carried out on the known cell cultures are only able to be reproduced during a very short time period of a few days, as the cell culture changes over the course of time.

A further disadvantage of cells which constantly divide is that the cells are therefore situated in a state which differs considerably from the situation in a tissue of an adult organism. A cell division in an adult body is a comparably very rare event. It takes place only after a loss of tissue and is accompanied as a rule by an inflammation reaction. Consequently, in these cells, the normal state is not being investigated, but in fact an extreme situation. This fact could explain why previous assay results which have been carried out using such cell cultures are often not consistent with clinical findings.

As it was previously not possible to keep dormant cells alive for a longer time, in contrast, a trend has developed to use particularly division-active cells for pharmacological assays and to shorten the observation time period. This, however, leads to the results being even less meaningful, as can be easily explained from the above statements.

### DISCLOSURE OF THE INVENTION

It is therefore the object of the present invention to create a method for producing a cell culture of human or animal cells to carry out medical and pharmacological assays with which a cell culture can be produced which is stable over a longer time period, in particular over several weeks. It is thereby possible to carry out medical or pharmacological assays on the same cell culture with an interval of several weeks. Additionally, it is an object of the present invention to create a cell culture which is produced by means of the method according to the invention and which is stable over a long time period, on which the desired medical or pharmacological assays can then be carried out.

The objects referred to are solved according to the invention by the features specified in the independent claims. Further embodiments of the invention result from the sub-claims.

According to the invention, a method for producing a cell culture of human or animal cells, in particular of mammalian cells, to carry out medical or pharmacological assays is proposed which comprises at least the following steps:
inserting a substrate of microbially produced cellulose into a vessel;
introducing a nutrient solution into the vessel;
applying human or animal cells to the substrate;
conditioning the vessel contents to a predetermined temperature, in particular body temperature;
waiting until the substrate is completely covered by a cell lawn, i.e. that all cells are touching and have stopped dividing (this can take a few days to several weeks, depending on the initial concentration and type of cell);
preferably, it is tested by means of metabolic parameters whether and when the cells have reached the state which is characteristic for them and
supplying an active substance to be tested on the cell culture located on the substrate.

After the application of the human or animal cells to the substrate of microcrystalline cellulose, a cell division and growth process occurs in which the substrate is covered increasingly by cells. This process takes place at body temperature, so at approximately 37° C. During the cell division and growth process, the cell culture is therefore preferably maintained at approximately 37° C. When the cells cover the substrate so thickly that the outer membranes of the cells are mutually touching without gaps, the cells stop dividing. After termination of the division process, the cells require a certain amount of time to convert and to reach a so-called dormant phenotype. Here, the expression “dormant” only refers to dramatic changes in shape no longer taking place as during the cell division. Biochemically, these cells are very active and produce, for example, factors which are typical for their differentiation state. In this state, the cell culture is stable on the substrate of microbially produced cellulose over at least several weeks.

With regard to the individual method steps of the method according to the invention it must be emphasized that this must not necessarily be executed in the specified order. Therefore, for example, the application of human or animal cells to the substrate can also take place before the introduction of a nutrient solution into the vessel. Optionally, the nutrient solution could also be introduced into the vessel first and only then could the substrate be inserted. It is clear to the person skilled in the art that he can specify the sequence of the individual steps within a certain scope according to desire.

The cells can be cooled slowly to room temperature of, for example, 15°-30° C., in particular 18°-25° C., for storage or for transport. It is not necessary to freeze the cell cultures for storage or for transport as they also remain stable at room temperature.

According to a preferred embodiment of the invention, the cell culture is preferably produced from stem cells or from cells which have been obtained from stem cells, such as, for example, liver, heart or kidney cells, or cells of other organs, such as, for example, the skin. A medical or pharmacological assay can therefore be carried out on known, precisely defined cells. In the case of cells obtained from stem cells, these cells can be produced, for example, with the aid of a method such as is known from US 20030161818A1 or US 2003 015 45 06 A1. Here, the stem cells are obtained from human umbilical cord tissue. The stem cells are located in the so-called substantia gelatinea funiculi umbilicalis, a gelatinous matrix rich in hyaluronic acid and chondroitin sulfates, also referred to as “Wharton's Jelly”. The extraction takes place via an enzymatic process (e.g. with the aid of collagenase).

Alternatively, such cells can of course also be used which have been removed directly from a human or animal body. This occurs, for example, via a biopsy of the bone marrow or of the fat tissue. According to one exemplary embodiment of the invention, for example, cells can be used which originate directly from affected patients, whereby a personalized therapy is possible.

The cells applied to the cellulose substrate are preferably such cells which have not previously already been passaged. The cell culture therefore results according to the invention by a single cell division and growth process of original, so-called primary cells, such as, for example, stem cells or cells obtained therefrom which have not undergone a dedifferentiation process. In other words, the cells applied to the cellulose substrate are preferably cells with a passage number equal to zero. Optionally, however, cells with a passage number of smaller than or equal to five could also be used.

It is not necessary to freeze the cells for transport, as is otherwise usual; rather, transport can take place at ambient temperature, such as, for example, between 15° C. and 30° C. The temperature can, however, also be higher or lower.

If the ambient temperature lies in the desired range, for example between 15° C. and 30° C., it is sufficient to allow the cell culture simply to cool (without using a cooling device). The cell culture could, however, also be cooled to the desired temperature actively, i.e. by means of a cooling device.

According to a preferred embodiment of the invention, the vessel is provided with a hermetic seal, such as, for example, a film or a cover. The hermetic seal in particular prevents the entry or exit of media, such as, for example, liquid or gas from or into the vessel. The sealing of the vessel can, in principle, be carried out before or after the cooling, but is preferably carried out prior to this.

According to a preferred embodiment of the invention, the vessel is sealed with a film, which, for example is fused or glued to the vessel. Alternatively or additionally, a cover can also be provided which seals the vessel.

The vessel having the cell culture located therein can finally be packaged further in order to be able to subsequently send it, for example, to a laboratory or to a client who then carries out the medical or pharmacological assays on the cell culture. The packaging can, for example, comprise a box and, if necessary, insulating material. The packaging can also comprise a coolant.

In order to protect the cell culture after the production thereof, in particular for a subsequent transport by post, or generally against mechanical loading and impact, it is proposed to provide a substance which increases the viscosity of the nutrient solution or a substance having a higher viscosity than the nutrient solution in the vessel. According to the first alternative, the nutrient solution, for example, can be offset with methyl cellulose, polyethylene glycol or another substance which increases the viscosity. According to the second alternative, a substance with a higher viscosity, such as, for example, a gel-like substance could also be introduced into the vessel. A solid, such as, for example, a body of microcrystalline cellulose could also be put on the cell culture. The cell culture is thereby mechanically secured on the base of the vessel and cannot tip heavily to the side or float free.

According to a preferred embodiment of the invention, the cells of the cell culture are not passaged, i.e. the cell lawn is preferably not detached and the cells are not separated as in prior art and then applied again to another substrate for new cell division. The cell culture is, rather, preferably produced by a single cell division and growth process of original cells, i.e. cells which have not previously been passaged.

The cell culture is preferably packaged in the same vessel in which it was grown and if necessary sent to a receiver. It therefore does not have to be decanted into another vessel.

According to a preferred embodiment of the invention, the method referred to above is carried out on a microwell plate which has a plurality of wells. A substrate of microbially produced cellulose is inserted into each of the wells, a nutrient solution is introduced and human or animal cells are applied to the substrate. Then the cells grow and separate until they have at least partially covered the substrate. Eventually, a part of the same cell culture is located in each of the individual wells of the microwell plate. The individual subcultures can then be used to carry out the same or different medical or pharmacological assays.

Storage or transport of the cell culture preferably takes place at ambient temperature. Freezing the cell culture, as has been usual until now, is not required.

The entire microwell plate is preferably provided with a hermetic seal, as is described above. It can finally be packaged and shipped.

The invention also relates to a microwell plate having several wells which are each filled with a substrate which is produced from microbial cellulose and which is populated by a cell culture. The microwell plate is preferably at approximately ambient temperature and is neither cooled nor heated. It could, however, also be cooled to temperatures below room temperature, for example to 5° C. to 10° C.

According to a preferred embodiment of the invention, a substance is contained in the individual wells of the microwell plate which is arranged on the cell culture and has a higher viscosity than the nutrient solution. The cell culture is thereby mechanically secured on the base of the vessel together with the substrate and cannot tip heavily to the side or float free.

Additionally, the invention also relates to a use of a cell culture of human or animal cells, in particular mammalian cells, which has been produced according to one of the methods described above, for carrying out medical or pharmacological assays.

The invention is explained in more detail by way of example by means of the included drawings. Here are shown:

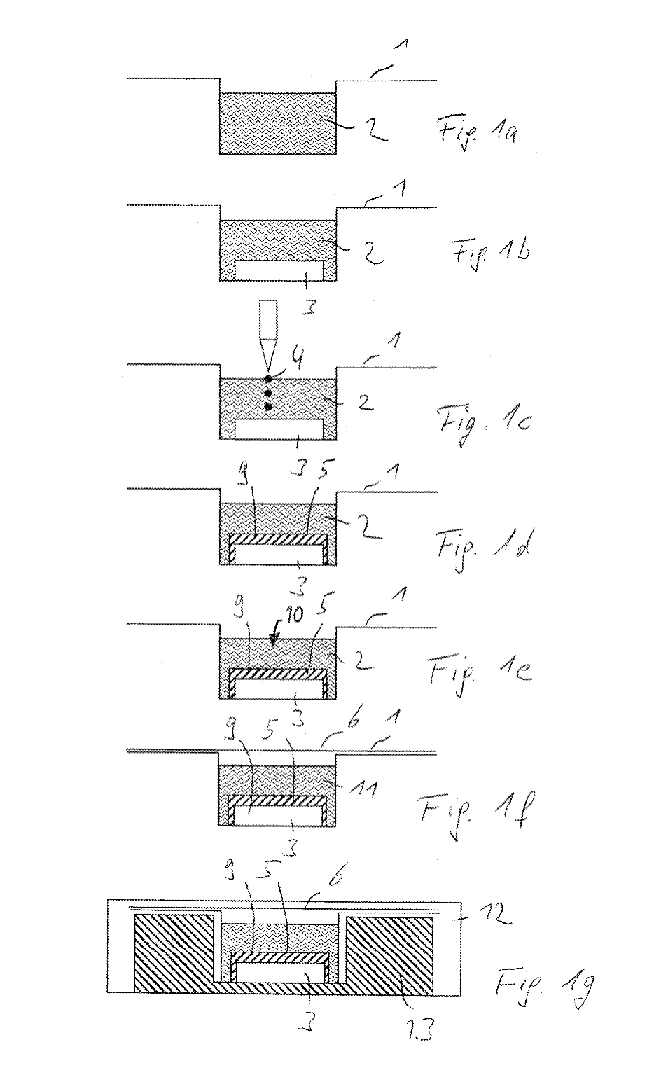

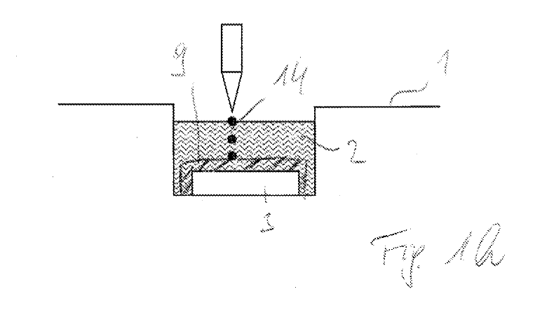

FIG. 1a-1h different states of a method for producing a cell culture of human or animal cells according to one embodiment of the invention; and

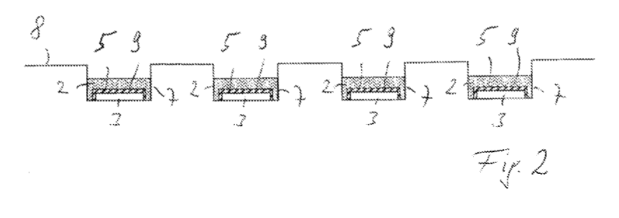

FIG. 2 a microwell plate having several wells in each of which a cell culture produced according to the method according to the invention is located.

FIGS. 1a-1h show different states of a method for producing a cell culture 9 of human or animal cells. The cell culture 9 can later be used to carry out medical or pharmacological assays.

To produce the cell culture 9, firstly a vessel 1 is filled with a nutrient solution 2 (FIG. 1a). For example, the nutrient medium described in the Biochemical Journal 58 from 1954, pages 345-352 by Schramm and Hestrin can be used as the nutrient solution 2. The nutrient solution can, for example, comprise 20 g glucose, 5 g yeast extract, 5 g Bacto Peptone, 2.7 g sodium phosphate and 1.15 g citric acid monohydrate and 0.5 g magnesium sulfate heptahydrate in a liter of water. Alternatively, other nutrient solutions known from prior art can be used.

As a next step, a substrate 3 which is produced from microbial cellulose and which can have, for example, the shape of a platelet is then put inside the vessel 1 (FIG. 1b). The two steps described above can also be carried out in reverse order. A substrate 3 of microcrystalline cellulose can, for example, be produced by means of a method such as is known from DE 10 2008 056 413.3 or WO 2010 052 019 A2. Other production methods to produce microcrystalline cellulose are sufficiently known from prior art.

In FIG. c, finally a suspension of human or animal cells 4 is introduced into the vessel 1. The cells applied to the substrate 3 can, for example, be stem cells or cells which have been obtained from stem cells, such as, for example, liver, heart or kidney cells or cells of other organs, such as, for example, the skin. The cells 4 can, for example, be produced with the aid of a method such as is known from US 02013025983A1, U.S. Pat. No. 6,410,320 B1 or U.S. Pat. No. 7,534,607 B1. Alternatively, such cells 4 can of course also be used which have been removed directly from a human or animal body.

The cells 4 applied to the cellulose substrate are preferably such cells which have not previously already been passaged. The cell culture 9 therefore results by a single cell division and growth process of original cells which have not undergone a dedifferentiation process. In order words, the cells 4 applied to the cellulose substrate 3 are preferably cells with a passage number equal to zero.

The cells 4 then sediment in the nutrient solution 2 and settle on the surface of the cellulose substrate 3. At temperatures around 37° C., a division and growth process then takes place, wherein a so-called cell lawn 5 forms on the substrate 3 within a few days, which covers the substrate 3 completely, as can be seen in FIG. 1d.

As soon as the cell lawn 5 has reached the desired size and thickness, the cell division and growth process stops. The vessel 1 including its contents is then cooled to a lower temperature. According to a preferred embodiment of the invention, the vessel is allowed to cool to room temperature, for example to approximately 18° C. to 25° C. It has been shown that, at these temperatures, the cell culture 9 remains stable over several weeks, for example over three, four or even more weeks, and the cells, in particular, do not die off. Depending on the type of cells, assays can be carried out on the cells, so, for example, addition of substances for the reformation of blood vessels in endothelial cells, the effect of possible toxic substances on liver cells, or the influence of the heart rate on heart muscle cells.

Optionally, in a further step le, an agent 10 which increases the viscosity of the nutrient solution 2, such as, for example, methyl cellulose or polyethylene glycol (PEG) can be added. The medium resulting after the addition of the agent 10 is marked in FIG. 1f with the reference numeral 11.

A substance having a higher viscosity than the nutrient solution or a solid can also be applied to the cell culture 9 instead of, or in addition to, the agent 10. Therefore, for example, a further body of microcrystalline cellulose could be put on the cell culture 9. It can thereby be prevented that the substrate 3 lifts from the base of the vessel 1 together with the cell culture 9, and, for example, tips to the side or floats free.

In a further step 1f, the vessel 1 can be sealed with a hermetic seal 6 in order to prevent the exit or entry of medium. The hermetic seal 6 can, for example, be a film, which, for example, can be fused or glued to the vessel 1. Optionally, a cover having a seal (not shown) could of course also be provided.

In order to ship the cell culture 9 including the vessel 1, the vessel 1 is finally packaged in step 1g. In the depicted exemplary embodiment, the vessel 1 is packed in a box 12 and additionally protected from mechanical impact and damage by a filler material 13. According to the invention, the cell culture 9 is preferably shipped in the same vessel 1 in which it was cultivated.

It is clear from the individual method steps 1a to 1g that it is not necessary to passage the cells of the cell culture 9, i.e. the cell lawn 5 is not detached from the substrate 3 and the cells are not separated and then applied again to another substrate for new cell division. The cell culture 9 is rather produced in a single growth and cell division process.

In FIG. 1h, it is finally depicted how a substance 14 to be tested is applied to the cell culture 9, while the cell culture 9 is furthermore located on the substrate 3. The reaction of the cells to the substance is then diagnosed.

FIG. 2 shows a microwell plate 8 having several wells 7 which each serve as a vessel 1 according to FIGS. 1a to 1g. A substrate 3 of microcrystalline cellulose is located in each of the wells 7, which is populated by a cell culture 9. The production of cell cultures 9 can therein be produced at the same time according to the method steps shown in FIGS. 1a to 1d.

After the individual cell cultures 9 have reached the desired size, the microwell plate 8 is cooled to room temperature and, for example, is sealed by means of a film 6, as is depicted in FIG. 1f. A part of the same cell culture is then located in each of the wells 7.

If the microwell plate 8 is to be shipped, it is packaged in a further step according to FIG. 1g. The client therefore obtains a microwell plate having a completed cell culture 9 which is stable over many weeks, on which he can carry out the desired medical or pharmacological assays.

1. A method to produce a cell culture of human or animal cells, in particular mammalian cells, to carry out medical or pharmacological assays, comprising the following steps:
inserting a substrate of microbially produced cellulose into a vessel;
introducing a nutrient solution into the vessel;
applying human or animal cells to the substrate;
conditioning the vessel contents to a predetermined temperature, preferably to approximately body temperature; and
waiting until the substrate is completely covered by a cell lawn, the cells no longer divide and have assumed a dormant phenotype,
wherein the cells applied to the cellulose substrate are such cells which have not previously been passaged and wherein the cell culture is produced in a single cell division and growth process.

2. The method according to claim 1, characterized in that the cell culture is cooled to approximately room temperature after the production thereof.

3. The method according to claim 1, characterized in that the cell culture is not frozen before carrying out a medical or pharmacological assay.

4. The method according to claim 1, characterized in that the cells applied to the substrate are stem cells or cells obtained from stem cells.

5. The method according to claim 1, characterized in that the cells applied to the substrate have not previously been passaged or have a passage number of less than or equal to five and in particular less than or equal to two.

6. The method according to claim 1, characterized in that the vessel is provided with a hermetic seal after producing the cell culture and before carrying out the medical or pharmacological assay.

7. The method according to claim 1, characterized in that the cells are not passaged, i.e. the cell lawn is not detached from the substrate, and the cells are not separated and then applied again to a substrate for new cell division.

8. The method according to claim 1, characterized in that the cell culture is packaged in the same vessel in which it was grown.

9. The method according to claim 1, characterized in that the method is carried out on several vessels at the same time.

10. The method according to claim 9, characterized in that the vessels are wells of a microwell plate.

11. The method according to claim 1, characterized in that a substance which increases the viscosity of the nutrient solution, or a substance having a higher viscosity then the nutrient solution, is additionally introduced into the vessel.

12. A microwell plate having several wells which are each filled with a substrate which is produced from microbial cellulose and which is populated by a cell culture, characterized in that the cell culture comprises cells which have not previously been passaged and the individual cells are hermetically sealed.

13. The microwell plate according to claim 12, characterized in that it additionally contains a substance which increases the viscosity of the nutrient solution or a substance having a higher viscosity than the nutrient solution, which is arranged on the cell culture.

14. A use of a cell culture of human or animal cells, in particular mammalian cells, which have been produced according to the method of claim 1, for carrying out medical or pharmacological assays.
